(19) United States
(12) Patent Application Publication
(10) Pub. No.: US 2008/0318221 A1
(43) Pub. Date: Dec. 25, 2008

(54) METHOD FOR THE DIAGNOSIS AND/OR PROGNOSIS OF ALZHEIMER'S DISEASE

(75) Inventors: Elisabet Rosell Vives, Barcelona (ES); Marta Barrachina Castillo, Barcelona (ES); Isidro Ferrer Abizanda, Barcelona (ES); Tamara Maes, Castelldefels (ES)

Correspondence Address: SUGHRUE MION, PLLC, 2100 PENNSYLVANIA AVENUE, N.W., SUITE 800, WASHINGTON, DC, 20037, US

(73) Assignees: FINA BIOTECH, S.L., POZUELO DE ALARCON (Madrid) (ES); ORYZON GENOMICS, S.A., BARCELONA (ES)

(21) Appl. No.: 11/765,714

(22) Filed: Jun. 20, 2007

(51) Int. Cl. C12Q 1/68; G01N 33/53

(52) U.S. Cl. 435/6; 435/7.1

(57) ABSTRACT

The present invention relates to a method for the diagnosis and/or prognosis of Alzheimer's disease by means of determining the DARC gene expression level in a biological sample and comparing said level with a reference value, in which the disturbance of said level is indicative of Alzheimer's disease.

### FIELD OF THE INVENTION

The present invention relates to the health sector, mainly to neurodegenerative diseases. More specifically, the invention is related to procedures for the diagnosis and/or prognosis of Alzheimer's disease.

### BACKGROUND OF THE INVENTION

Alzheimer's disease (AD) is considered the main cause of dementia, this being the fourth cause of death in developed countries (Pappolla M A. La Neuropatología y la Biología Molecular de la Enfermedad de Alzheimer (Neuropathology and Molecular Biology of Alzheimer's disease). pp 543-553. Neuropathology. Diagnosis and Clinical Medicine. Cruz-Sánchez FF. Ed. Edimsa. 2000.). It is defined as a central nervous system neurodegenerative condition and is characterised by a progressive deterioration of higher cerebral functions.

Microscopically, AD is characterised by the presence of senile plaques (diffuse and classic), neurofibrillary tangles, neuropil threads, neuronal degeneration, β-Amyloid protein deposits, granulovacuolar degeneration and the presence of Hirano bodies, amongst other pathologies (Cruz-Sánchez FF et al. Neuropathological Diagnostic Criteria for Brain banking. Ed. IOS Press. 1995).

Clinical criteria that are well established in the fourth edition of the diagnostic and statistical manual of the American Psychiatric Association (DSM-IV) are used to diagnose AD (Diagnostic criteria from DSM-IV. Washington DC: APA; 1994) or by the National Institute of Neurologic, Communicative Disorders and Stroke—Alzheimer's Disease and Related Disorders Association (NINCDS-ADRDA), (Mc Khann G, Drachman D, Folstein M, Katzman R, Price D, Stadlan E M. “Clinical diagnosis of Alzheimer's Disease: report of the NINCDS ADRDA work group under the auspices of Department of Health and Human Services Task Force on Alzheimer's Disease. Neurology 1984; 34:939-44). Nevertheless, the greatest dilemma for these clinical studies is diagnostic certainty. Although AD is diagnosed by means of several neurological tests, currently the only way to confirm the diagnosis is performing a post-mortem analysis in brain tissue in order to find the existence of neurofibrillary tangles and plaques.

Different genetic markers have been studied in recent years for application in AD diagnosis, such as:
the determination of mutations in the amyloid precursor protein (APP) gene, mutations in the presenilin-1 (PS1) and presenilin-2 (PS2) genes, only valid for a reduced number of cases of precocious or family AD (Gil Nécija, Eulogio. Biological diagnosis. Fourth National Course on Alzheimer's Disease. Seville, 23-24 Sep. 1999. Ed. Andrómico).
genetic value of the ApoE genotyping, ascertained only in those cases complying with probable AD clinical criteria; the problem is that it gives a high number of false positives.

As well as these genetic markers, there are biochemical markers such as:
the Tau protein: this protein is ascertained by means of neuronal antibodies capable of detecting tau in cerebrospinal fluid, however, tau levels in AD are not related to age, sex, disease development, nor with the degree of dementia, as well as high levels of tau being detected in other pathologies such as meningitis, meningeal infiltrations, frontal lobe dementia and Creutzfeldt-Jacobs disease.
the β-Amyloid protein: this protein lacks diagnostic utility in sporadic forms of AD (Guierá A. et al. “Update on the pathology of Alzheimer's disease”. Rev Esp Patol 2002; Vol 35, n^{o} 1:21-48.).

AD presents a pre-symptomatic stage, without definite clinical symptoms, which may last between 10 and 20 years. There is currently no non-intrusive diagnostic tool available with suitable sensitivity, specificity and predictive value for this disease. Moreover, this disease implies huge social costs, due, among other reasons, to the incapacity by the patients to cope for themselves, this leading to the necessity of a reliable diagnostic procedure in pre-clinical stages that allows preventing the disease, improving the treatment and predicting disease development.

To this effect, the authors of the present invention have surprisingly found that the DARC gene expression level is clearly higher in biological samples from patients with Alzheimer's disease compared to reference values from control samples.

Chemokines are a type of cytokines with chemotactic activity (hence the name) which direct leukocyte migration and are involved in a wide range of physiological and pathological processes, mainly in immunitary and inflammatory processes. They are low molecular weight proteins (approximately 70 aminoacids) secreted by different cells and involved in leukocyte migration and activation, in angiogenesis processes, in collagen production and in the proliferation of hematopoietic precursors.

Their action is carried out via interaction with their specific receptors that are expressed on the cell surface, a subgroup of transmembrane receptors coupled to protein G. These receptors are promiscuous, being therefore capable of binding different chemokines and thus producing different biological effects.

It is known that some chemokine receptors play a role in the pathogenesis or susceptibility to infectious diseases. Among these, the DARC receptor (erythrocyte Duffy antigen or “Duffy blood group, chemokine receptor”) has the ability to bind members of the CXCL and CCL chemokine subfamilies. The ligand-receptor bond causes internalisation but does not lead to signal transduction. Seemingly, DARC plays a regulatory role consisting in preventing chemokine-mediated inflammatory damage. Furthermore, it is considered useful for their transport and depuration in circulation. This receptor is expresed in red blood cells, postcapillary venule endothelium, Purkinje cells (cerebellum) and activated T lymphocytes.

On the other hand, the DARC receptor is a cofactor for malaria parasites Plasmodium vivax and Plasmodium knowlesi entry into erythrocytes. Resistance to P. vivax in malaria has been described as associated with a lack of expression of the DARC receptor.

Nevertheless, there is no evidence to date on the relationship between this gene and Alzheimer's Disease.

Based on this finding, and the requirements of the state of the art, the authors of the present invention have developed a simple and reliable procedure for the diagnosis and/or prognosis of AD based on detecting DARC gene expression levels.

The use of the DARC gene as a genetic marker for AD allows establishing an early diagnosis of the disease in pre-clinical stages, as well as a prognosis of the development thereof.

### DESCRIPTION OF THE DRAWINGS

FIG. 1: Amplification curves for the DARC gene corresponding to hippocampus samples from patientes suffering Alzheimer's disease I and III and control patients (C). The curves with the lowest Ct (threshold cycle) (left of the image) correspond to pathological samples, wherease the two curves appearing at higher Ct (right of the image) correspond to control patient samples.

FIG. 2: Amplification curves for the DARC gene corresponding to entorhinal area samples from patientes suffering Alzheimer's disease III and control patients (C). The curves with the lowest Ct (threshold cycle) (left of the image) correspond to pathological samples, wherease the two curves appearing at higher Ct (right of the image) correspond to control patient samples.

FIG. 3: Amplification curves for the DARC gene corresponding to neocortex samples from patientes suffering Alzheimer's disease I, II and V and control patients. The curves with the lowest Ct (threshold cycle) (left of the image) correspond to pathological samples, wherease the two curves appearing at higher Ct (right of the image) correspond to control patient samples.

FIG. 4: Analysis of protein expression in brain tissue using the Western Blot technique with a specific anti-DARC antibody and using beta-actin as load control. Images of the bands obtained from A) frontal cortex in AD I (upper left), AD III (upper right), and B) in entorhinal cortex in AD I (lower left) and AD III (lower right).

FIG. 5: Specific staining using an anti-DARC antibody. Immunohistochemistry of hippocampus sections. Left: tissue from an Alzeimer's disease patient (A: AD), right: tissue from an individual not suffering Alzeimer's disease (B: Control)

FIG. 6: Specific staining using an anti-DARC antibody. Immunohistochemistry of frontal cortex sections. Left: tissue from an Alzeimer's disease patient (A: AD), right: tissue from an individual not suffering Alzeimer's disease (B: Control)

### OBJECT OF THE INVENTION

Firstly, the invention relates to a method for the diagnosis and/or prognosis of Alzheimer's Disease based on determining DARC gene expression level in biological samples.

Also object of the present invention is a kit for the diagnosis and/or prognosis of AD to carry out the determination of DARC gene expression level according to the previous procedure.

### DESCRIPTION OF THE INVENTION

The present invention provides a method for the diagnosis and/or prognosis of Alzheimer's Disease by means of determining DARC gene expression level.

A main aspect of the invention relates to a method for the diagnosis and/or prognosis of Alzheimer's Disease in a subject by determining the DARC gene expression level in a biological sample isolated from the subject and by comparing said level with a reference value, where the alteration of said level is indicative of Alzheimer's disease and/or of the stage of said disease.

Thus, said procedure may be performed with a diagnostic purpose (diagnostic method) and with a prognosis purpose (prognosis method). A diagnostic procedure relates to a procedure that allows determining genes that are differentially expressed between samples of Alzheimer's disease patients and control samples (from healthy individuals). The prognostic method relates to a method that allows predicting, at least in part, disease development by means of analysing the differential expression of said genes in the different stages of the disease. In this sense a subject who has been previously diagnosed with AD might be analyzed to know the progress of the disease.

The term “subject” used in the present invention relates to a human being.

The expression “reference value” in the present invention designates mRNA or DARC protein levels present in a healthy individual not suffering from AD or other diseases affecting mRNA or DARC protein levels.

According to a particular embodiment of the invention, the biological sample comprises a tissue, preferably said tissue is a tissue homogenate, preferably the tissue homogenate is obtained from nervous tissue cells or peripheral neuroendocrine cells.

According to another particular embodiment of the invention, the biological sample is a biological fluid, preferably said biological fluid preferably comprises cerebrospinal fluid, blood, plasma or serum.

In a particular embodiment of the invention, the determination of DARC gene expression level is performed by analysing the amount of RNA or protein coded by said gene or fragments thereof.

Particularly, the determination of DARC gene expression level is performed by image analysis. In preferred embodiments, the image analysis may be carried out from quantification on immunohistochemical images. Examples of quantification methods include, but are not limited to, morphometry, densitometry and fluorescence intensity.

In a particular embodiment of the invention, the determination of DARC gene expression level is carried out by means of an indicator substance that binds specifically to the RNA or the protein coded by said gene.

In the present invention, the expression “indicator substance” refers to an antibody, a monoclonal antibody, an antibody fragment, an oligonucleotide, a macromolecule, an organic molecule, or in general, any substance that may bind specifically to RNA or the protein coded by the DARC gene. Said indicator substance comprises a marker which may be an enzyme, a radioisotope, a dye, a fluorescent compound, a chemoluminescent compound, a bioluminescent compound, a metal chelate or, generally, any known marker of the state of the art that may be detected by a detection method.

According to another particular embodiment of the invention, determination of the DARC gene expression level is performed by analysing the amount of RNA coded by said gene or fragments thereof.

In a preferred embodiment of the invention, the analysis of the amount of RNA coded by said gene or fragments thereof is performed by amplification, using oligonucleotides specific to PCR, SDA or any other amplification method for cDNA allowing a quantitative estimation of DARC transcript levels.

In another preferred embodiment of the invention, the analysis of the amount of RNA coded by said gene or fragments thereof is performed by means of DNA biochips made with oligonucleotides deposited by any mechanism known by a person skilled in the art or synthesised in situ by means of photolithography or by means of any other mechanism known by a person skilled in the art.

In another particular embodiment of the invention, the determination of the expression level for the gene coding for DARC is performed by analysing the amount of protein coded by said gene or fragments thereof.

In a preferred embodiment of the invention, the analysis of the amount of protein coded by said gene or fragments thereof is performed by Western-Blot.

In another preferred embodiment of the invention, the analysis of the amount of protein coded by said gene or fragments thereof is performed by means of protein chips using specific antibodies against DARC or fragments thereof or by protein profiles performed by mass spectrometry or by any other mechanism allowing a quantitative estimate of DARC protein levels.

In another preferred embodiment of the invention, the analysis of the amount of protein coded by said gene or fragments thereof is performed by immunohistochemical techniques.

In another preferred embodiment of the invention, the analysis of the amount of protein coded by DARC or fragments thereof is performed by incubation with a specific antibody.

In another preferred embodiment, the analysis of the amount of protein coded by said gene is performed by means of ELISA or any other enzymatic method.

Another main aspect of the invention is a kit for the diagnosis and/or prognosis of Alzheimer's Disease comprising the reagents necessary for carrying out the determination of DARC gene expression level. The Kit allows carrying out the method according to the invention that has just been described.

In a particular embodiment of the invention, the diagnosis and/or prognosis kit for AD comprises the reagents necessary for determining the DARC gene expression level by means of image analysis.

In another particular embodiment, the reagents necessary to determine the DARC gene expression level comprise a composition comprising an indicator substance that binds specifically to the RNA or the protein coded by said gene, where said indicator substance is marked with a detectable marker and a physiologically acceptable carrier liquid.

In a particular embodiment of the invention, the diagnosis and/or prognosis kit for Alzheimer's disease comprises the reagents necessary for determining the level of RNA coded by the DARC gene.

In a preferred embodiment of the invention, the diagnosis and/or prognosis kit for Alzheimer's disease comprises the reagents necessary for determining the level of RNA coded by the DARC gene and/or fragments thereof by amplification.

In another preferred embodiment of the invention, the diagnosis and/or prognosis kit for Alzheimer's disease comprises the reagents necessary for determining the level of RNA coded by the DARC gene and/or fragments thereof by DNA biochips.

In another particular embodiment of the invention, the diagnosis and/or prognosis kit for Alzheimer's disease comprises the reagents necessary for determining the level of protein coded by the DARC gene and/or fragments thereof.

In another preferred embodiment of the invention, the diagnosis and/or prognosis kit for Alzheimer's disease comprises the reagents necessary for determining the level of protein coded by the DARC gene and/or fragments thereof by Western-Blot.

In another preferred embodiment of the invention, the diagnosis and/or prognosis kit for Alzheimer's disease comprises the reagents necessary for determining the level of protein coded by the DARC gene and/or fragments thereof by protein chips.

In another preferred embodiment of the invention, the diagnosis and/or prognosis kit for Alzheimer's disease comprises the reagents necessary for determining the level of protein coded by the DARC gene and/or fragments thereof by immunohistochemical techniques.

In another preferred embodiment of the invention, the diagnosis and/or prognosis kit for AD comprises the reagents necessary for analysing the amount of protein coded by the DARC gene and/or fragments thereof by incubation with a specific antibody.

Finally, a preferred embodiment of the invention contemplates a diagnosis and/or prognosis kit for AD comprising the reagents necessary for analysing the amount of protein coded by the DARC gene and/or fragments thereof by ELISA or any other enzymatic procedure.

Other aspects of the invention will become evident for a person of average skill in the art.

The following examples serve to illustrate but not limit the present invention.

### EXAMPLES

### Example 1

### Initial Determination of Genes Differentially Expressed in Samples from Patients with AD

An experiment was performed on DNA micromatrices in order to identify genes that had differential expression levels between brain tissue samples from Alzheimer patients and controls. Neocortex and hippocampus tissues from several donors in development stages I/II and II/IV were used for the study, compared with normal expression in normal tissue from material extracted from the same areas. Especially, the DARC gene was determined as an overexpressed gene in Alzheimer disease patients.

Brain samples were obtained by autopsy of 12 patients with AD and 6 controls. Informed consent was obtained from the patients or their relatives and the study was approved by the Ethics Committees.

The time between death and tissue processing was 2-10 hours. Half the brain was cut in 1 cm thick coronal sections and was frozen in dry ice at −80° C. until use.

For the morphological examination, the brains were fixed by immersion in a 10% formalin buffer during at least 48 hours at 4° C.

The neuropathological study was carried out in 4 μm paraffin sections without wax of the upper frontal cortex, anterior convolution of the corpus callosum, frontal white matter from the semioval centre, occipital lobe white matter from semioval centre, head of caudate nucleus and nucleus accumbens, nucleus of Meynert, lenticular nucleus, anterior thalamus, medial central thalamus, dorsal thalamus, hippocampus, lower temporal convolution and amygdaloid nucleus, anterior insula, pre- and post-central convolution, calcarine convolution, mesencefalon at the level of the substantia nigra, high protuberance at the level of the cerulen loci, low protuberance, medulla oblongata, spinal chord, spinal ganglions, cerebellar vermis, cerebellar hemisphere and dentate nucleus, optic chiasm and olfactory bulb.

The sections were stained with hematoxylin and eosin, Luxol Fast Blue by the Klüver Barrera procedure and for glial fibre acid protein immunohistochemistry, CD 68 and tomato lectin for microglia, β-Amyloid, pan-tau, specifically phosphorylated tau at Thr181, Ser202, Ser214, Ser262, Ser396 and Ser422 and αB-crystallin, α-sinuclein and ubiquitin.

AD stages were established according to amyloid load and according to neurofibrillary pathology following the Braak and Braak classification:

Stage A: initial deposits in the basal neocortex

Stage B: deposits extended to the association areas of the neocortex

Stage C: strong deposits throughout the entire cortex

I-II: neurofibrillary pathology stages in transentorhinal region

III-IV: limbic region

VI: neocortical region

Having prepared the samples, both DARC mRNA and protein mRNA were determined and biochemical, immunohistochemical and microscopic studies were performed. The samples of control and diseased brains were processed in parallel. The results from these studies demonstrat that there is an increase in DARC and protein mRNA expression levels in the cerebral cortex in early stages of AD. The DARC protein is clearly overexpressed in Alzeimer's disease patients, localised in nuclei in neurons and cytoplasm in astrocytes.

### Example 2

### mRNA Isolation and Confirmation of Results by cDNA Synthesis and TaqMan PCR

Quantitative PCR with specific probes is the technique usually used as a reference for validating changes in gene expression detected by oligonucleotide micromatrices. 20 independent tissue samples were used for the validation, from the entorhinal area (8 controls, 5 ADI and 7ADIII), 19 hippocampus samples (6 controls, 7 ADI and 6 ADIII) and 28 neocortex samples (7 controls, 8 ADI, 7 ADIII, 6 ADV).

Total RNA was isolated using Trizol Reagent® (Life Technologies) followed by RNeasy Protect Mini Kit (Qiagen). Frozen human cerebral tissue were homogenised directly in 1 ml of Trizol per 100 mg of tissue. Total RNA was extracted following the protocol suggested by the supplier. The purified Total RNA was then resuspended in 100 μl of RNase-free water. mRNA was purified following the RNeasy Protect Mini Kit protocol with minimal modifications. Treatment with DNase was dismissed due to the elimination of genomic DNA during extraction with Trizol. The concentration of each sample was measured at A₂₆₀, and RNA integrity was verified by formaldehyde-agarose gel electrophoresis and by bioanalyzer analysis.

The samples selected for the analysis were chosen very strictly regarding RNA quality. Degradation is a parameter which clearly influences obtaining a reliable result or otherwise a result that hardly allows quantifying the expression level.

The High-Capacity cDNA Archive Kit (Ref 4322171) by Applied Biosystems was used for cDNA synthesis. Calibration curves were obtained starting from 2.5 μg of RNA which were passed to cDNA and for the remaining samples the synthesis was performed starting from 1 μg of total RNA. For each 100 μl of reverse transcription reaction, the human RNA was mixed with the master mix provided by the retailer containing: random hexamers, MgCl 2, 500 μM each of dATP, dTTP, dCTP and dGTP, 0.4 U/μl of RNase inhibitor and 1.25 U/μl of transcriptase in the appropriate buffer. reactions were carried out at 25° C. for 10 minutes in order to maximise bonding between the template RNA and the primer, followed by 120 min at 37° C. and after by incubation for 5 min at 95° C. in order to deactivate the reverse transcriptase.

TaqMan low density Arrays-Microfluidic Cards by Applied Biosystems were used to validate 20 genes that were differentially expressed in the DNA micromatrices performed with postmortem cerebral tissue samples from patients with Alzheimer's disease. The endogenous controls incorporated in the Microfluidic Cards were the GUS (β-glucuronidase) and 18S (18S ribosomal subunit) genes.

The TaqMan probe (Applied Biosystems) binds to the template DNA strand between the direct and reverse primers. The probe containes a fluorescent molecule and another molecule capable of screening the first molecule's fluorescence if it is close enough. If there is a specific reaction, the probe is degraded by the Taq polymerase during amplification, releasing the fluorescent molecule, which separates from its screening molecule thus emitting fluorescence. The amount of fluorescence produced will therefore be proportional to the amount of product accumulated.

The TaqMan PCR tests for DARC and the internal controls were performed in duplicate on cDNA samples on the multifluidic cards. Parallel tests were performed for each sample using β-actin and GUS primers and probes for standardisation. The reaction was carried out using the following parameters: 50° C. for 2 minutes, 95° C. for 10 minutes and 40 cycles at 95° C. for 15 seconds and 60° C. for 1 minute. Standard curves were prepared for DARC and for each internal control using serial dilutions of control samples of human RNA. Finally, the TaqMan PCR data was captured using Sequence Detector Software (SDS version 1.9; Applied Biosystems).

It must be taken into account that in order to perform a relative quantification, the expression of a particular gene in a sample is standardised with respect to an endogenous gene of invariable expression. The lines obtained for the endogenous gene and the genes to be studied when representing the threshold cycle (Ct) with respect to the amount of cDNA used. ABI 7700 measures fluorescence accumulation by the PCR products by continuous monitoring. The detection threshold is fixed after the reaction. The detection threshold is an arbitrary value manually assigned to a level above the baseline in the exponential PCR phase in which there is no limiting element. The Ct value establishes the point at which sample amplification crosses the detection threshold. The levels of the internal controls used to normalise DARC mRNA values did not vary in the pathological samples with respect to the controls and were also similar between the different pathologies. In order to apply the relative quantification procedure comparing Cts (delta-delta Ct procedure) it is necessary that the lines obtained from the gene to be studied and from the gene used as an endogenous gene are parallel. The relative amount will be defined by the following formula:

2^{-ΔΔCt}

Due to the low expression of the DARC gene in brain tissue of individuals not affected by Alzheimer's disease it was not possible to determine Ct in the control samples. Nevertheless, the difference in Ct confirms great overexpression.

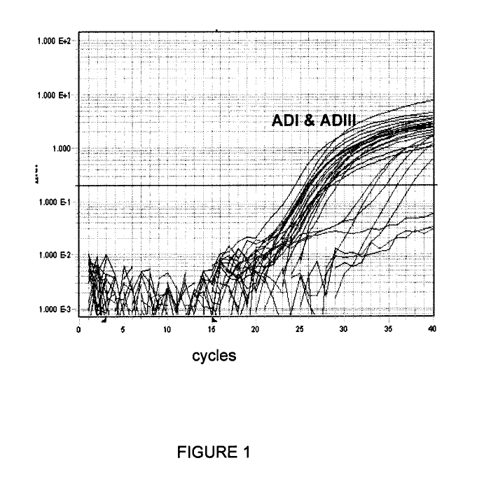

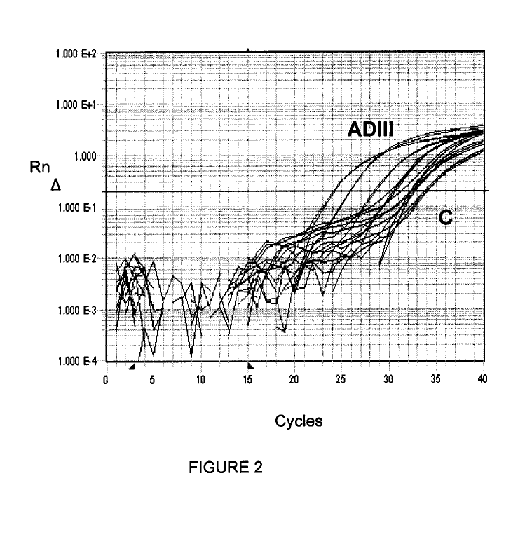

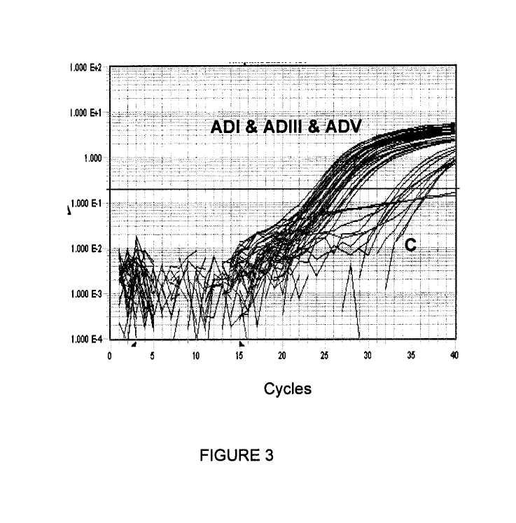

The increase in DARC mRNA levels was confirmed by means of TaqMan PCR tests with the control brain samples and with AD in hippocampus tissue (FIG. 1), entorhinal area (FIG. 2) and neocortex (FIG. 3). The results showed an increase in DARC mRNA levels in the hippocampus, the entorhinal area and the neocortex of tissues belonging to AD patients in states I and II when compared to controls. The levels of this gene in the control tissues were low. It was therefore not possible to calculate relative quantification of patient samples with respect to control tissues. The importance of the fact that the expression of this gene is low in healthy tissues and clearly increases in tissues affected by Alzheimer's disease still in clinical symptom stages (stages I and II) is worth mentioning.

### Example 3

### Electrophoresis Gel and Western Blot Analysis

The frozen frontal cortex and entorhinal cortex samples (about 100 mg) were directly homogenised in 1 ml of lysis buffer (20 mM Hepes, 10 mM KCl, 1.5 mM MgCl₂, 1 mM EDTA, 1 mM EGTA, 1 mN DDT, 2 mM PMSF, 1 μg/ml aprotinin, leupeptin and pepstatin) and sonicated. The lysates were centrifuged at 5.000 rpm for 10 minutes at 4° C. and protein concentration was determined by Pierce's BCA procedure. 20 μg of total proteins were subjected to 95° C. and then loaded into SDS-polyacrylamide gels with a Tris-glycine elution buffer.

The proteins were subjected to electrophoresis using a Mini-Protean system (Bio-Rad) and they were transferred to nitrocellulose membranes (Bio-Rad) with a Mini Trans Blot electrophoresis transfer cell (Bio-Rad) for 1 hour at 100 V. The nitrocellulose membranes were blocked with phosphate buffered saline solution (PBS) with 0.1% of Tween 20 (TBST) containing 5% of skimmed milk, for 60 minutes. The membranes were then incubated at 4° C. overnight with one of the primary antibodies in TBST with 3% bovine serum albumin (BSA). The following antibodies were used: goat anti-human DARC polyclonal antibody: goat anti-duffy/DARC antibody (EB06940, Everest Biotech) 1:500 dilution, anti-β-actin antibody (AC-74 clone, Sigma) 1:5000 dilution. After incubation with the primary antibody, the membranes were washed three times with TBST for 5 minutes at room temperature and they were then incubated with goat and mice anti-IgG antibodies marked with radish peroxidase (Dako) at a 1:1000 dilution (1:5000 for β-actin) for one hour at room temperature. The membranes were then washed 4 times, 5 minutes each time with TBST at room temperature and developed by the ECL Western Blot chemoluminiscence system (Amersham/Pharmacia), being subsequently exposed to autoradiography film (Hyperfilm ECL, Amersham).

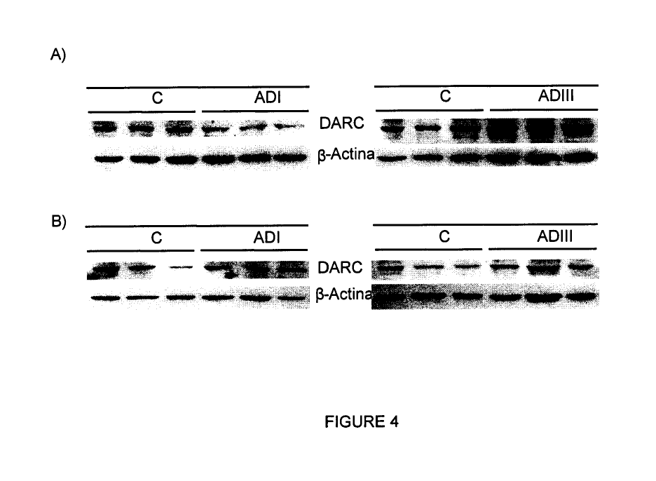

The results showed that the DARC protein levels, as DARC mRNA levels, were increased in the frontal cortex in AD III stages (FIG. 4).

### Example 4

### Immunohistochemistry

The 5 μm thick sections without wax from the frontal cortex, hippocampus and frontal cortex were processed for immunohistochemistry following the streptavidin-biotin peroxidase (LSAB) procedure. After incubation with methanol and H₂O₂ in PBS and normal serum, the sections were incubated with goat anti-human DARC polyclonal antibody (EB06940, Everest Biotech) in a 1:250 dilution. After incubation with the primary antibody, the sections were incubated with LASB for 15 minutes at room temperature. The peroxidase reaction was visualised with diaminobenzidine and H₂O₂. Immunostaining control included omitting the primary antibody. No signal was obtained following incubation exclusively with the secondary antibody. The sections were slightly stained with hematoxilin.

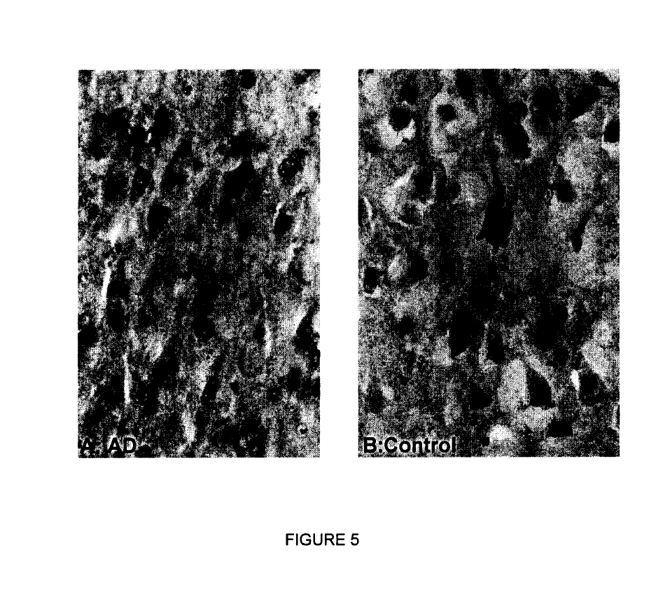

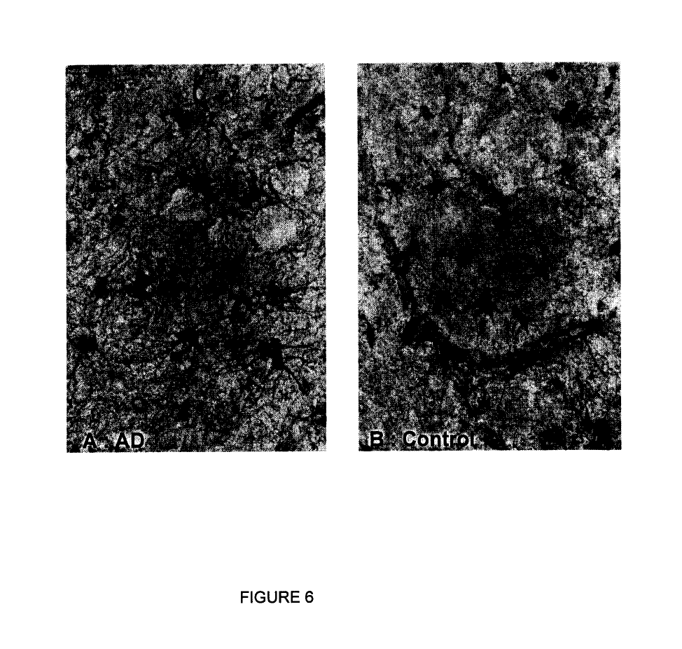

DARC's immunoreactivity characterised by small cytoplasm granules was mainly located in astrocytes when observing frontal cortex tissues (FIG. 5). In the hippocampus samples the DARC protein was mainly located in neurons, showing nuclear localisation (FIG. 6). Expression was clearly greater in Alzheimer's disease patient tissue than in control samples.

1. A method for the diagnosis and/or prognosis of Alzheimer's Disease (AD) comprising:
determining the expression level of the DARC gene in a biological sample isolated from a subject, and
comparing said expression level with a reference value,
in which the alteration of said level is indicative of AD and/or the stage of said disease.

2. A method for the diagnosis or prognosis of AD according to claim 1, in which the biological sample comprises a tissue.

3. A method for the diagnosis and/or prognosis of AD according to claim 2, in which the tissue is a homogenate.

4. A method for the diagnosis and/or prognosis of AD according to claim 3, in which the tissue homogenate is obtained from nervous tissue cells or from peripheral neuroendocrine cells.

5. A method for the diagnosis and/or prognosis of AD according to claim 1, in which the biological sample comprises a biological fluid.

6. A method for the diagnosis and/or prognosis of AD according to claim 5, in which the biological fluid is cerebrospinal fluid, blood, plasma or serum.

7. A method for the diagnosis and/or prognosis of AD according to claim 1, in which the determination of the DARC gene expression levels performed by analysing the amount of RNA or protein coded by said gene or fragments thereof.

8. A method for the diagnosis and/or prognosis of AD according to claim 7, in which the in accordance with gene expression level is determined by image analysis.

9. A method for the diagnosis and/or prognosis of AD according to claim 7, in which the determination of the DARC gene expression level is performed by means of an indicator substance which binds specifically to the RNA or protein coded by said gene.

10. A method for the diagnosis and/or prognosis of AD according to claim 7, in which the determination of the DARC gene expression level is performed by analysing the amount of RNA coded by said gene or fragments thereof.

11. A method for the diagnosis and/or prognosis of AD according to claim 10, in which the analysis of the amount of RNA is performed by amplification.

12. A method for the diagnosis and/or prognosis of AD according to claim 10, in which the analysis of the amount of RNA is performed by DNA biochips.

13. A method for the diagnosis and/or prognosis of AD according to claim 7, in which the determination of the DARC gene expression level is performed by analysing the amount of protein coded by said gene and/or fragments thereof.

14. A method for the diagnosis and/or prognosis of AD according to claim 13, in which the analysis of the amount of protein is performed by Western Blot.

15. A method for the diagnosis and/or prognosis of AD according to claim 13, in which the analysis of the amount of protein is performed by protein chips.

16. A method for the diagnosis and/or prognosis of AD according to claim 13, in which the analysis of the amount of protein is performed by immunohistochemical techniques.

17. A method for the diagnosis or prognosis of AD according to claim 13, in which the analysis of the amount of protein is performed by incubation with a specific antibody.

18. A method for the diagnosis or prognosis of AD according to claim 13, in which the analysis of the amount of protein is performed by ELISA or any other enzymatic procedure.

19. A Kit for the diagnosis and/or prognosis of AD comprising the reagents necessary for determining the DARC gene expression level according to the method of claim 1.

20. A Kit for the diagnosis and/or prognosis of AD comprising the reagents necessary for determining the DARC gene expression level by image analysis, according to the method of claim 8.

21. A Kit for the diagnosis and/or prognosis of AD comprising a composition containing an indicator substance that binds specifically to RNA or to the protein coded by said gene, in which said indicator substance is marked with a detectable marker, and a physiologically acceptable carrier liquid, according to the method of claim 9.

22. A Kit for the diagnosis and/or prognosis of AD comprising the reagents necessary for the amplification of the RNA coded by the DARC gene and/or fragments thereof, according to the procedure of claim 11.

23. A Kit for the diagnosis and/or prognosis of AD comprising the reagents necessary for analysing the RNA coded by the DARC gene and/or fragments thereof by means of DNA biochips, according to the method of claim 12.

24. A Kit for the diagnosis and/or prognosis of AD comprising the reagents necessary for analysing the amount of protein coded by the DARC gene and/or fragments thereof by Western Blot, according to the method of claim 14.

25. A Kit for the diagnosis and/or prognosis of AD comprising the reagents necessary for analysing the amount of protein coded by the DARC gene and/or fragments thereof by protein chips, according to the method of claim 15.

26. A Kit for the diagnosis and/or prognosis of AD comprising the reagents necessary for analysing the amount of protein coded by the DARC gene and/or fragments thereof by immunohistochemical techniques, according to the method of claim 16.

27. A Kit for the diagnosis and/or prognosis of AD comprising the reagents necessary for analysing the amount of protein coded by the DARC gene and/or fragments thereof by incubation with a specific antibody, according to the method of claim 17.

28. A Kit for the diagnosis and/or prognosis of AD comprising the reagents necessary for analysing the amount of protein coded by the DARC gene and/or fragments thereof by ELISA or any other enzymatic procedure, according to the method of claim 18.
